(19) United States
(12) Patent Application Publication
(10) Pub. No.: US 2024/0337643 A1
(43) Pub. Date: Oct. 10, 2024

(54) AGE DIFFERENTIATION OF CRUDE OILS USING CHEMICAL FOSSIL ASSEMBLAGE

(71) Applicant: ExxonMobil Technology and Engineering Company, Spring, TX (US)

(72) Inventors: Chun Zhu, The Woodlands, TX (US); Muhammad Asif, The Woodlands, TX (US); Zhibin Wei, Sugar Land, TX (US)

(73) Assignee: ExxonMobil Technology and Engineering Company, Spring, TX (US)

(21) Appl. No.: 18/294,642

(22) PCT Filed: Jul. 25, 2022
(86) PCT No.: PCT/US2022/074108

§ 371 Date: Feb. 2, 2024

Related U.S. Application Data
(60) Provisional application No. 63/260,214, filed on Aug. 12, 2021.

(51) Int. Cl. G01N 33/28; E21B 43/00; E21B 44/00

(52) U.S. Cl. CPC G01N 33/2823; E21B 43/00; E21B 44/00

(57) ABSTRACT

Age differentiation of hydrocarbon samples may be achieved using a chemical fossil assemblage approach. For example, a method may comprise: determining a source facies for a hydrocarbon sample; inputting the source facies into a chemical fossil assemblage model; determining, using the chemical fossil assemblage model, one or more candidate chemical fossil assemblages and corresponding age biomarkers for the hydrocarbon sample based on the source facies; measuring a concentration or a related value of corresponding age biomarkers in the hydrocarbon sample to yield an age biomarker fingerprint; inputting the age biomarker fingerprint into a chemical fossil assemblage model; comparing the age biomarker fingerprint to the one or more candidate chemical fossil assemblages using the chemical fossil assemblage model; and estimating an age of the hydrocarbon sample based on the comparison.

### CROSS-REFERENCE TO RELATED APPLICATION

This application claims the benefit of U.S. Provisional Patent Application 62/260,214 filed 12 Aug. 2021 entitled AGE DIFFERENTIATION OF CRUDE OILS USING CHEMICAL FOSSIL ASSEMBLAGE, the entirety of which is incorporated by reference herein.

### FIELD OF THE INVENTION

The present disclosure relates to differentiate the geological age of liquid hydrocarbons (also referred to herein as oil). The geological age of a liquid hydrocarbon, as used herein, refers to the depositional age of the source rock from which the liquid hydrocarbons are derived.

### BACKGROUND

Source rocks are organic rich sediments and are the origin of hydrocarbons accumulated in the subsurface. Source rocks are often not penetrable because such rocks are at a great depth. However, sedimentary organic matter signatures preserved in source rocks is transformed into hydrocarbons with increasing temperature and pressure. As illustrated in FIG. 1, the hydrocarbons generated in the source rocks, driven by pressure and buoyancy, migrate through carrier beds and accumulate in sealed geological traps that are more accessible.

Analogous to physical fossils preserved in sediments, oils contain numerous chemical compounds encoding the generative source rock information. More specifically, when an organism fossilizes the physical fossil remains in the source rock and chemicals associated with said physical fossil (or chemical fossils) can leach into the oil. Therefore, just as physical fossils are aged, the chemical fossils can be used to correlate the migrated hydrocarbons to the deeply buried source rocks, from which the oils are derived. Such chemical fossils are also known as age biomarkers.

For example, angiosperm plants have flowers and produce seeds enclosed within a carpel. As the earth evolved through the Late Cretaceous (100-65 million years before present, or 100-65 Ma BP) epoch through the Paleocene (65-55 Ma BP), Eocene (55-34 Ma BP), Oligocene (34-23 Ma BP), and Miocene (23-5 Ma BP) epochs, angiosperm progressively dominated the higher plant community. Therefore, the presences of angiosperm-specific biomarkers as chemical fossils of angiosperm in oil can be used to constrain the oil age of Late Cretaceous or younger (i.e., <100 Ma BP). More specifically, oleanane is one of the most commonly used angiosperm-specific biomarkers because oleanane survives degradation and is easily analyzed using gas spectroscopy-mass spectroscopy (GC-MS) to identify the presence and concentration of oleanane. For age differentiation, a relative concentration of oleanane (defined as the concentration ratio of oleanane to hopane (H30)) higher than 0.03 indicates an age of Cretaceous-Tertiary, or <100 Ma BP as described in “The Molecular Fossil Record of Oleanane and Its Relation to Angiosperms,” Science 635, 768-777 (1994). However, an age resolution of <100 Ma BP based on the presence of oleanane is often insufficient to address the hydrocarbon origin in many petroleum basins and makes pinpointing the stratigraphic depth of the corresponding source rocks almost impossible.

### SUMMARY OF THE INVENTION

The present disclosure relates to age differentiation of hydrocarbon samples using a chemical fossil assemblage approach

A nonlimiting example method of the present disclosure may comprise: determining a source facies for a hydrocarbon sample; inputting the source facies into a chemical fossil assemblage model; determining, using the chemical fossil assemblage model, one or more candidate chemical fossil assemblages and corresponding age biomarkers for the hydrocarbon sample based on the source facies; measuring a concentration or a related value of corresponding age biomarkers in the hydrocarbon sample to yield an age biomarker fingerprint; inputting the age biomarker fingerprint into a chemical fossil assemblage model; comparing the age biomarker fingerprint to the one or more candidate chemical fossil assemblages using the chemical fossil assemblage model; and estimating an age of the hydrocarbon sample based on the comparison.

Another nonlimiting example method of the present disclosure may comprise: determining a source facies for a hydrocarbon sample; measuring a concentration or a related value of age biomarkers in a hydrocarbon sample to yield an age biomarker fingerprint; inputting the source facies and the age biomarker fingerprint into a chemical fossil assemblage model; comparing the age biomarker fingerprint to one or more facies-dependent chemical fossil assemblages using the chemical fossil assemblage model; and estimating an age of the hydrocarbon sample based on the comparison.

A nonlimiting example system of the present disclosure may comprise: a computing device comprising: a processor; a memory coupled to the processor; and instructions provided to the memory, wherein the instructions are executable by the processor to perform the chemical fossil assemblage model steps of either or both of the methods.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates the components of a hydrocarbon system in a subsurface region. These components are effective source rock, thermal maturation, reservoir rocks, migration pathway, seal (cap rock), and trap.

FIG. 2 illustrates a plurality of partial chromatograms of seed-plants derived molecular fossils including Oleanane (A), Bicadinanes (B), Simonellite and Retene (C), and Diterpanes (D-G).

FIG. 3 is a flow diagram of a nonlimiting example analytical workflow for ascertaining characteristics of a hydrocarbon sample.

FIG. 4 is a flow diagram of a nonlimiting example age prediction method using a chemical fossil assemblage approach of the present disclosure.

FIG. 5 is a flow diagram of another nonlimiting example age prediction method using a chemical fossil assemblage approach of the present disclosure.

FIG. 6 is a nonlimiting example of chemical fossil assemblage.

FIG. 7 is a nonlimiting example of statistical constraint and differentiation of oil ages using the molecular assemblage approach.

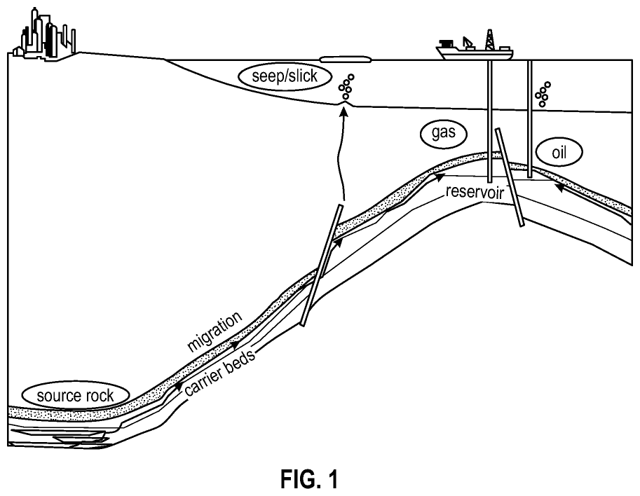

### DETAILED DESCRIPTION

To facilitate a better understanding of the embodiments of the present invention, the examples of preferred or representative embodiments are given within the detailed description. In no way should the following examples be read to limit, or to define, the scope of the invention.

The present disclosure relates to age differentiation of hydrocarbon samples using a chemical fossil assemblage approach. More specifically, the chemical fossil assemblage approach includes a correlation to the concentration of two or more chemical fossils (also referred to as age biomarkers) to source rock age. Said correlation may be facies-dependent. That is, the facies describes the rock characteristics imparted by the formation, composition, and fossil content of said rock. Examples of facies include, but are not limited to, marine carbonate and marly facies, marine clastic and deltaic facies, paralic facies, lacustrine facies, terrestrial and coaly facies, paralic facies, and the like.

Generally, the methods and systems described herein include determining the facies of the source rock (also referred to herein as source facies) for a hydrocarbon sample and then aging the hydrocarbon sample based on the concentration of chemical fossil (also referred to herein as age biomarkers) in the hydrocarbon sample using predictive models trained using a database of known sample ages and chemical fossil compositions. Without being limited by theory, it is believed that concentration of an age biomarker in a hydrocarbon sample depends on, among other things, the source facies and the age of the source rock. The methods and systems described herein advantageously account for such dependencies, which may provide higher accuracy of aging hydrocarbon samples.

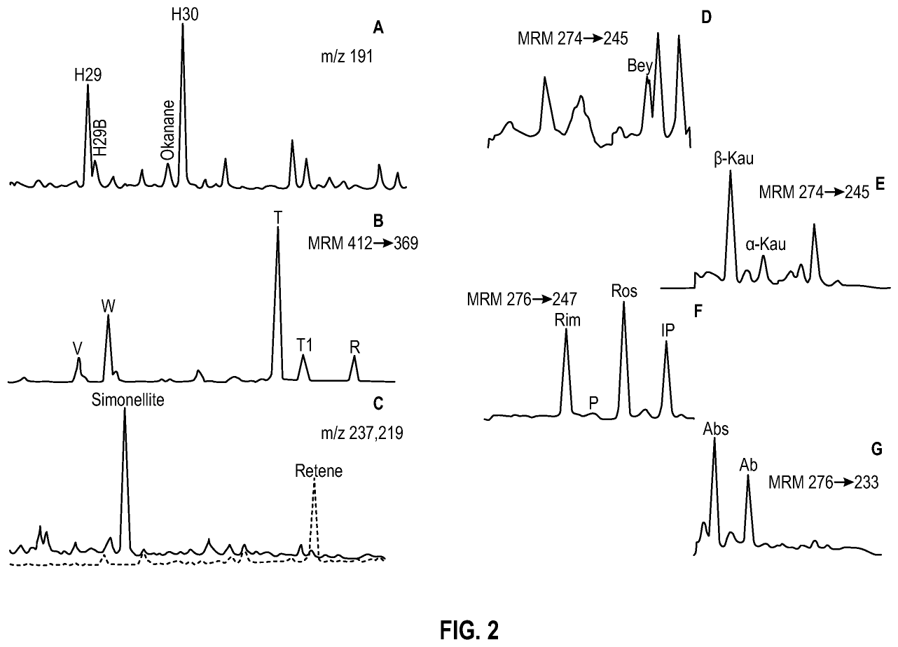

FIG. 2 illustrates a plurality of partial chromatograms of seed-plants derived molecular fossils including Oleanane (A), Bicadinanes (B), Simonellite and Retene (C), and Diterpanes (D-G) where H29 refers to 30-nor-hopane; H29 Ts refers to 30-nor-neohopane; H30 refers to C30 hopane; V, W, T, T1, R refer to bicadinanes and derivatives; Bey refers to beyerane; β-Kau refers to β-Kaurane; α-Kau refers to α-Kaurane; Rim refers to Rimuane; Ros refers to Rosane; P refers to Pimerane; IP refers to IsoPimerane; Ab refers to abietane; and Abs refers to abietarosane.

The systems and methods described herein may be able to age hydrocarbon samples using the chemical fossil assemblage approach. For example, samples may be differentiated into various epochs including, but not limited to, Upper Triassic (237-200 Ma BP), Lower Jurassic (200-175 Ma BP), Middle Jurassic (175-161 Ma BP), Upper Jurassic epoch (161-145 Ma BP), Lower Cretaceous (145-100 Ma BP), Late Cretaceous (100-65 Ma BP), Paleocene (65-55 Ma BP), Eocene-Oligocene (55-23 Ma BP), and Miocene and younger (23-0 Ma BP), and the like, and stages (sub-epochs) thereof, and any combination of abutting epochs. Examples of stages include, but are not limited to, Tithonian (152-145 Ma BP) and Kimmeridgian (157-152 Ma BP). For example, the systems and methods described herein using the chemical fossil assemblage approach may differentiate the age of a hydrocarbon sample between Upper Triassic (237-200 Ma BP), Lower Jurassic (200-175 Ma BP), and Middle/Upper Jurassic (175-145 Ma BP). In another example, the systems and methods described herein using the chemical fossil assemblage approach may differentiate the age of a hydrocarbon sample between Upper Triassic (237-200 Ma BP), Lower Jurassic (200-175 Ma BP), Middle Jurassic (175-161 Ma BP), Upper Jurassic epoch (161-145 Ma BP), Lower/Late Cretaceous (145-65 Ma BP), Paleocene (65-55 Ma BP), Eocene-Oligocene (55-23 Ma BP), and Miocene and younger (23-0 Ma BP). In yet another example, the systems and methods described herein using the chemical fossil assemblage approach may differentiate the age of a hydrocarbon sample between Jurassic or older epoch (237-145 Ma BP), Lower Cretaceous (145-100 Ma BP), Late Cretaceous (100-65 Ma BP), Paleocene (65-55 Ma BP), Eocene (55-34 Ma BP), and Oligocene/Miocene epoch (34-5 Ma BP).

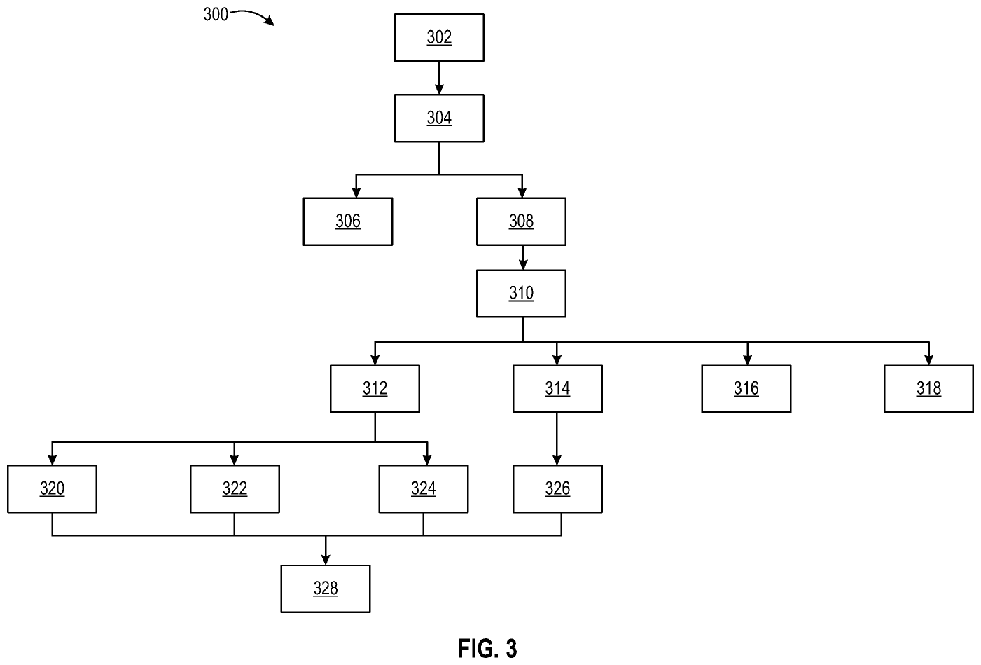

Because crude oils are a complex mixture of natural products, the oil samples may need to be fractionated to remove compounds that interfere the detection of age biomarkers. FIG. 3 is a nonlimiting example flow diagram of a sample analytical flowchart 300 to ascertain the identity and concentration of chemical fossils (or age biomarker fingerprint) in the oil sample. The sample 302 (e.g., crude oil, seep oil, oil stain, or rock extract) with unknown age may be directly analyzed by gas chromatography (GC) 304. Alternatively, an extract of the sample may be analyzed by GC 304. The biodegradation level is determined based on the GC results.

Severely-biodegraded samples 306 may not be further analyzed by the chemical fossil assemblage approach.

Un-biodegraded to moderately-biodegraded samples 308 (e.g., having a labile organic matter of 8 to 11) may proceed to further analysis for age differentiation by the chemical fossil assemblage approach. The next step in the illustrated flowchart 300 is fractionation 310 into a plurality of fractions 312, 314, 316, and 318. For example, the asphaltenes 318 may be extracted from the sample 308 using n-pentane. The remaining portion of the sample 308 may be separated into saturates fraction 312, aromatic fraction 314, and polar fraction 316 using high-pressure liquid chromatography (HPLC). The saturates fraction 312 may be analyzed by gas chromatography-isotope ratio mass spectrometry (GC-irMS) 320, gas chromatography/mass spectrometry (GC/MS) 322, and gas chromatography/mass spectrometry tandem mass spectrometry (GC/MS/MS) 324 to ascertain at least a portion of the identity and concentration of chemical fossils (or age biomarker fingerprint) 328 in the saturates fraction 312. The aromatics fraction 314 from the HPLC separation is also analyzed by GC-MS 326 to ascertain at least a portion of the identity and concentration of chemical fossils (or age biomarker fingerprint) 328 in the aromatics fraction 314.

Other secondary analyses (not illustrated) may be performed on the original sample 302 and/or fractions 312, 314, 316, and 318 thereof to ascertain a level of biodegradation, a level of contamination, sample maturity, sulfur content, American Petroleum Institute (API) gravity, the structures of chemical fossils, and other characteristics of the hydrocarbon sample. One skilled in the art will recognize suitable analysis techniques for measuring such characteristics. Nonlimiting examples of analysis technique include: GCxGC-TOFMS (time of flight mass spectroscopy), time of flight mass spectroscopy, whole oil gas chromatography, 13C isotopic composition, full-scan gas chromatography/mass spectroscopy, infrared-gas chromatography/mass spectroscopy, C₄ to C₁₉ gas chromatography, and the like, and any combination thereof.

Any of the foregoing analytical techniques can be replaced or augmented with comparable techniques known to one of skill in the art.

As described further herein, the chemical fossil assemblage approach may use predictive models trained using data from samples with known age and source rock and measured at least a portion of identity and concentration of chemical fossils (or age biomarker fingerprint). Said age biomarker fingerprint data may be collected using the description in FIG. 3 or a variation thereof. Further, samples with unknown age may be characterized by the method described in FIG. 3 or a variation thereof where the data is then used by the predictive model to estimate an age of the sample.

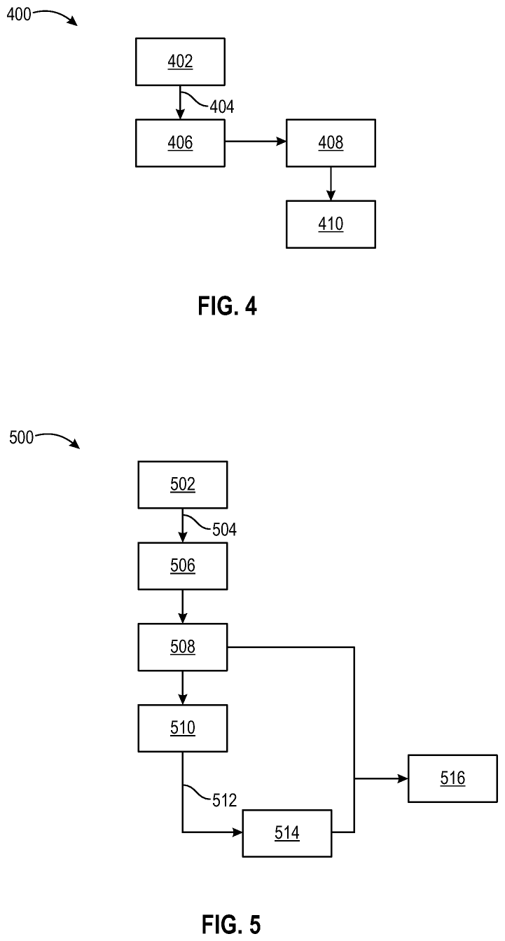

For example, FIG. 4 is a nonlimiting example flow diagram of a method 400 for age differentiation using a chemical fossil assemblage approach. A hydrocarbon sample with unknown age 402 may first processed through an analysis workflow 404 (e.g., the workflow or a portion thereof illustrated in FIG. 3) to determine the characteristics 406 of the source rock of the hydrocarbon sample 402. Said characteristics 406 may include the source facies and other properties like the maturity and/or alteration of the hydrocarbon sample. For example, the analysis workflow 404 may be FIG. 3 or a portion thereof. For example, the source facies and maturity may be determined from the GC-MS 322 and GC-MS/MS 324 results of the saturates fraction 312 and the GC-MS 326 results of the aromatics fraction 314.

The characteristics 406 (e.g., facies, maturity, at least a portion of an age biomarker fingerprint, and other characteristics) of the hydrocarbon sample 402 may then be input to a chemical fossil assemblage model 408.

A chemical fossil assemblage model (or approach) of the present disclosure may be a predictive model (e.g., a random forest decision tree, a neural network, or the like) that is trained using data for hydrocarbon samples having known a known age and at least following characteristic known: source rock, maturity, at least a portion of an age biomarker fingerprint, and other characteristics.

Random Forest classifier may be used to predict the age of crude oils. The distribution of age biomarker assemblages as well as maturity and facies data in samples with known ago are used as predictors that formulate the training dataset for supervised machine learning. Each decision tree is subsequently grown on a bootstrap sample of the training dataset. A bootstrap sample is a random sample draw from the training dataset with replacement. For a classification or regression tree, the Mean Decrease Gini (MDG) is applied for classification algorithm that searches all possible split of predictors to best predict the response (i.e., known age of the oil in the training sample set). The predictions of all trees are finally aggregated through majority voting. The importance of predictors may be evaluated by the out-of-bag error or impurity. Prior to application, the accuracy of Random Forest prediction is assessed by validation sample set.

Referring back to FIG. 4, the chemical fossil assemblage model 408 may estimate one or more ages 410 (or produce one or more estimated ages 410) for the hydrocarbon sample with unknown age 402 based on the analysis of the characteristics 406 of said sample 402. Each estimated age 410 may have a corresponding value that relates to the probability or other metric that indicates how likely the estimated age 410 is correct.

FIG. 5 illustrates another nonlimiting example flow diagram of a method 500 for age differentiation using a chemical fossil assemblage approach. A hydrocarbon sample with unknown age 502 may first processed through an analysis workflow 504 (e.g., the workflow or a portion thereof illustrated in FIG. 3) to determine the characteristics 506 of the source rock of the hydrocarbon sample 502. Said characteristics 506 may include the source facies and other properties like the maturity and/or alteration of the hydrocarbon sample. For example, the analysis workflow 504 may be FIG. 3 or a portion thereof. For example, the source facies and maturity may be determined from the GC-MS 322 and GC-MS/MS 324 results of the saturates fraction 312 and the GC-MS 326 results of the aromatics fraction 314.

The characteristics 506 (e.g., facies, maturity, and other characteristics) of the hydrocarbon sample 502 may then be input to a chemical fossil assemblage model 508. In this example, the age biomarker fingerprint is not yet considered by the chemical fossil assemblage model 508. The chemical fossil assemblage model 508 may output, based on the at least the facies and maturity, the age biomarkers 510 best suited for ascertaining an age of the hydrocarbon sample with unknown age 502. That is, the chemical fossil assemblage model 508 may determine which chemical fossils (or age biomarkers) need to be analyzed for abundance based on the characteristics 506. For example, an age biomarker present in marine carbonate and marly facies may not be present at all or in significant quantities for age differentiation when considering paralic facies. Accordingly, if the facies is determined to be a paralic facies, the chemical fossil assemblage model 508 may not tell a user to analyze the hydrocarbon sample with unknown age 502 for said age biomarker. Whereas, if the facies is determined to be a marine carbonate and marly facies, the chemical fossil assemblage model 508 may tell a user to analyze the hydrocarbon sample with unknown age 502 for said age biomarker.

Once the age biomarkers 510 are designated by the chemical fossil assemblage model 508, the age biomarker fingerprint 514 or a portion thereof may be measured 512 based on the age biomarkers 510 designated by the chemical fossil assemblage model 508. In some instances, some or all of the age biomarker fingerprint 514 may have been previously measured. Accordingly, if needed, additional aspects of the age biomarker fingerprint 514 may be measured based on the age biomarkers 510 designated by the chemical fossil assemblage model 508. Then, the age biomarker fingerprint 514 may be input to the chemical fossil assemblage model 508 to estimate one or more ages 516 (or produce one or more estimated ages 516) for the hydrocarbon sample with unknown age 502 based on the analysis of the characteristics 506 and age biomarker fingerprint 514 of said sample 502. Each estimated age 516 may have a corresponding value that relates to the probability or other metric that indicates how likely the estimated age 516 is correct.

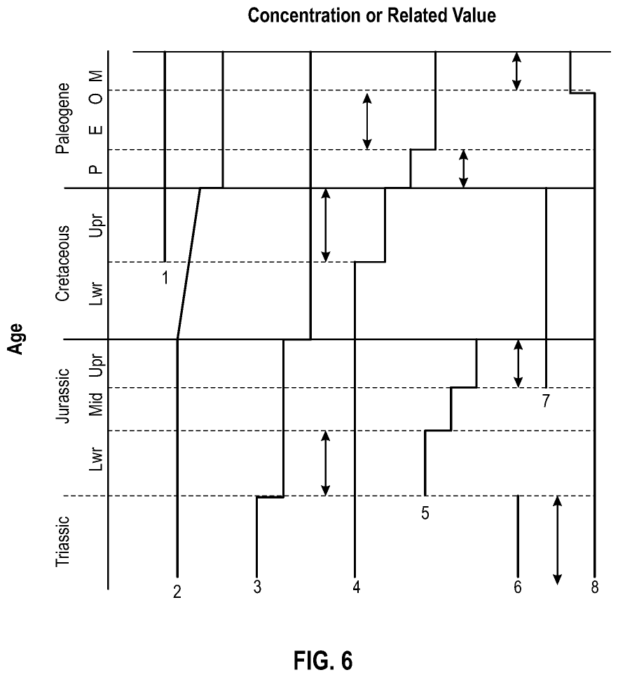

FIG. 6 is a nonlimiting example of chemical fossil assemblage may be applicable to marine carbonate and marly facies. Along the x-axis 1-8 are age biomarkers biosynthesized by gymnosperms, angiosperms (including both C3 and C4 plants), dinoflagellates, diatoms, and bacteria. In the illustrated example, the concentrations of each are illustrated with the highest concentration being toward the left of the illustration and lower concentrations to the right of the illustration. Further, the concentration levels along the lines are generalized as trend lines and vary along the trend line with age. That is, this is a simplified illustration of a chemical fossil assemblage.

Each age biomarker concentration is illustrated in FIG. 6 independently. That is, the left to right location of the line indicating the concentration of age biomarker 1 in no way relates to the left to right location of the line indicating the concentration of any other age biomarkers.

A nonlimiting example method of the present disclosure may include inputting the characteristics like facies, optionally maturity, and age biomarker fingerprint for a hydrocarbon sample into a chemical fossil assemblage model described herein. The chemical fossil assemblage model may use the facies and maturity to select the chemical fossil assemblage of FIG. 6 and then use the age biomarker fingerprint to estimate the age of the hydrocarbon sample. Alternatively, FIG. 6 may be one of a plurality of chemical fossil assemblage considered by the chemical fossil assemblage model. The chemical fossil assemblage model may output one or more estimated ages of the hydrocarbon sample, optionally with a metric for the likelihood said age is correct.

In another nonlimiting example method, the facies and optionally maturity may be input to the chemical fossil assemblage model. FIG. 6 may be the only chemical fossil assemblage selected or one of a plurality of chemical fossil assemblages selected by the chemical fossil assemblage model as the candidate chemical fossil assemblage(s). The chemical fossil assemblage model may then output the age biomarkers to be measured. For example, if FIG. 6 is the only chemical fossil assemblage, the chemical fossil assemblage model may output that age biomarkers 1-8 should be measured in the hydrocarbon sample. If FIG. 6 is one of many candidate chemical fossil assemblages, the chemical fossil assemblage model may output that age biomarkers 1-8 as well as other age biomarkers from the other candidate chemical fossil assemblages should be measured in the hydrocarbon sample (e.g., additionally age biomarkers 9-12 where age biomarkers 1-8 may independently also be present in said other candidate chemical fossil assemblages). Individual age biomarkers may be present in more than one chemical fossil assemblage. In either instance, the concentration or abundance of the age biomarkers provided by the chemical fossil assemblage model may be measured and input into the chemical fossil assemblage model. The chemical fossil assemblage model may then output one or more estimated ages of the hydrocarbon sample, optionally with a metric for the likelihood said age is correct.

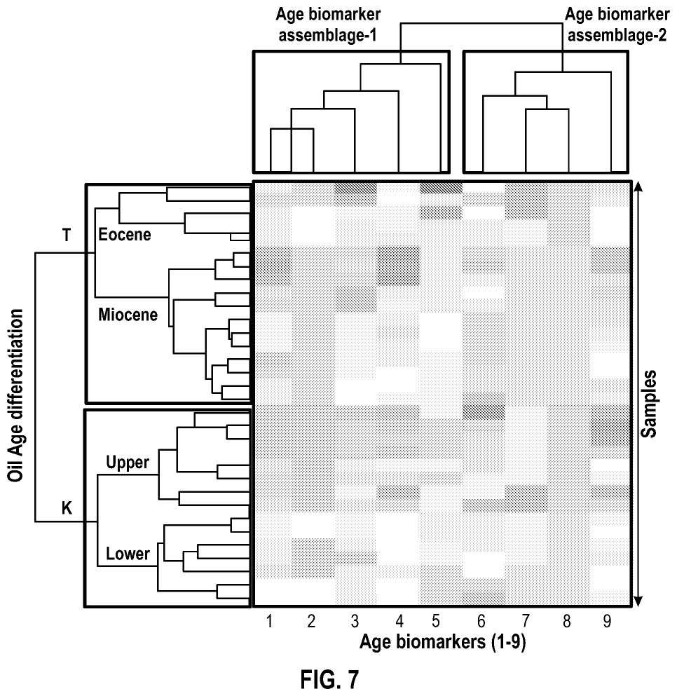

FIG. 7 is a nonlimiting example of statistical constraint and differentiation of hydrocarbon ages using the molecular assemblage approach. More specifically, two age biomarker assemblages are illustrated as columns and each row is a sample of a specific age.

Various aspects of the systems and methods described herein utilize computer systems. Such systems and methods can include a non-transitory computer readable medium containing instructions that, when implemented, cause one or more processors to carry out the methods described herein.

“Computer-readable medium” or “non-transitory, computer-readable medium,” as used herein, refers to any non-transitory storage and/or transmission medium that participates in providing instructions to a processor for execution. Such a medium may include, but is not limited to, non-volatile media and volatile media. Non-volatile media includes, for example, NVRAM, or magnetic or optical disks. Volatile media includes dynamic memory, such as main memory. Common forms of computer-readable media include, for example, a floppy disk, a flexible disk, a hard disk, an array of hard disks, a magnetic tape, or any other magnetic medium, magneto-optical medium, a CD-ROM, a holographic medium, any other optical medium, a RAM, a PROM, and EPROM, a FLASH-EPROM, a solid state medium like a memory card, any other memory chip or cartridge, or any other tangible medium from which a computer can read data or instructions. When the computer-readable media is configured as a database, it is to be understood that the database may be any type of database, such as relational, hierarchical, object-oriented, and/or the like. Accordingly, exemplary embodiments of the present systems and methods may be considered to include a tangible storage medium or tangible distribution medium and prior art-recognized equivalents and successor media, in which the software implementations embodying the present techniques are stored.

The methods described herein can be performed using computing devices or processor-based devices that include a processor; a memory coupled to the processor; and instructions provided to the memory, wherein the instructions are executable by the processor to perform the methods described herein. The instructions can be a portion of code on a non-transitory computer readable medium. Any suitable processor-based device may be utilized for implementing all or a portion of embodiments of the present techniques, including without limitation personal computers, networks of personal computers, laptop computers, computer workstations, mobile devices, multi-processor servers or workstations with (or without) shared memory, high performance computers, and the like. Moreover, embodiments may be implemented on application specific integrated circuits (ASICs) or very large scale integrated (VLSI) circuits.

### Applications of Oil Aging

The methods and systems described herein may be useful in distinguishing the age of hydrocarbon samples. As such, the methods and systems described herein may provide a better understanding of the origin of oils, oil stains, or oil seeps, which allows one to de-risk petroleum basins and better differentiates exploration opportunities.

In a first nonlimiting example, the specific age of oils, oil stains, or oil seeps ties the samples to a specific stratigraphic interval (or source rock) where hydrocarbons are derived. As a result, hydrocarbon migration pathways from the source to trap can be illustrated. The migration pathway may determine whether source rocks can effectively charge the location (e.g., a trap, a basin, or the like) from which the hydrocarbon sample was obtained to form economic hydrocarbon accumulation. Therefore, in some instances, once the source rock and migration pathway has been determined, a wellbore may be drilled into the location (e.g., a trap, a basin, or the like) from which the hydrocarbon sample was obtained. When referring to location here, the exact location is not implied by rather a general location of the trap, the seep, the basin, or the like that would contain oil from the same source rock as the hydrocarbon sample.

In another nonlimiting example, the hydrocarbon generation timing can be calculated based on the specific oil age and the thermal history of the basin. The resultant generation timing is further compared with the timing of trap emplacement. If the trap is deposited earlier than hydrocarbon generation, the source rock is likely to charge the trap and it is considered highly risky to form economic hydrocarbon accumulation.

Other applications of aging and the benefits of better resolved aging per the methods and systems described herein will be apparent to those skilled in the art.

The new method is capable of distinguishing Late Cretaceous vs. Paleocene vs. Eocene or younger sourced oils. It improves age resolution compared with published age biomarker resolution of Late Cretaceous or younger. As such, the new method helps with better understanding of the origin of oils, oil stains, or oil seeps, which allows to de-risk petroleum basins and better differentiates exploration opportunities.

In a first nonlimiting example, the specific age of oils or oil stains, or oil seeps ties the samples to a specific stratigraphic interval where hydrocarbons are derived. As a result, hydrocarbon migration pathways from the source to trap can be illustrated. This is critical because the migration pathway determines whether source rocks can effectively charge the reservoir to form economic hydrocarbon accumulation.

In another nonlimiting example, the hydrocarbon generation timing can be calculated based on the specific oil age and the thermal history of the basin. The resultant generation timing is further compared with the timing of trap emplacement. If the trap is deposited earlier than hydrocarbon generation, the source rock is likely to charge the trap and it is considered highly risky to form economic hydrocarbon accumulation.

Other applications of aging and the benefits of better resolved aging per the methods and systems described herein will be apparent to those skilled in the art.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of“comprising” various components or steps, the compositions and methods can also “consist essentially of” or “consist of” the various components and steps.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces.

1. A method comprising:
determining a source facies for a hydrocarbon sample;
inputting the source facies into a chemical fossil assemblage model;
determining, using the chemical fossil assemblage model, one or more candidate chemical fossil assemblages and corresponding age biomarkers for the hydrocarbon sample based on the source facies;
measuring a concentration or a related value of corresponding age biomarkers in the hydrocarbon sample to yield an age biomarker fingerprint;
inputting the age biomarker fingerprint into the chemical fossil assemblage model;
comparing the age biomarker fingerprint to the one or more candidate chemical fossil assemblages using the chemical fossil assemblage model; and
estimating an age of the hydrocarbon sample based on the comparison.

2. The method of claim 1, wherein the source facies is selected from the group consisting of: a marine carbonate/marly facie, a marine clastic/deltaic facie, a terrestrial/coaly facie, and a lacustrine facie.

3. The method of claim 1, wherein the chemical fossil assemblage model is a predictive model trained using a database of known sample ages and age biomarker compositions.

4. The method of claim 1, wherein the measuring of the concentration or the related value of the age biomarkers:
separating the hydrocarbon sample into separated fractions including an aromatics fraction and a saturates fraction;
analyzing the saturates fraction using one or more of: gas chromatography-isotope ratio mass spectrometry, gas chromatography/mass spectrometry, and gas chromatography/mass spectrometry tandem mass spectrometry; and
analyzing the aromatics fraction using gas chromatography-mass spectrometry.

5. The method of claim 1, further comprising:
performing one or more analyses selected from: time of flight mass spectroscopy, whole oil gas chromatography, ¹³C isotopic composition, full-scan gas chromatography/mass spectroscopy, infrared-gas chromatography/mass spectroscopy, and C₄ to C₁₉ gas chromatography.

6. The method of claim 1, wherein the determining of the source facies for the hydrocarbon sample is performed using an extract of the hydrocarbon sample.

7. The method of claim 1, further comprising:
identifying a source rock of the hydrocarbon sample based on the age of the hydrocarbon sample; and
estimating a migration pathway for a hydrocarbon source of the hydrocarbon sample based on the source rock and a location from which the hydrocarbon sample was obtained.

8. The method of claim 1, further comprising:
estimating risk for drilling a wellbore and producing hydrocarbon from the wellbore in the location from which the hydrocarbon sample was obtained based on the age of the hydrocarbon sample and a thermal history of the location from which the hydrocarbon sample was obtained.

9. The method of claim 1, further comprising:
drilling a wellbore into a location from which the hydrocarbon sample was obtained.

10. The method of claim 1, further comprising:
producing hydrocarbon from a location from which the hydrocarbon sample was obtained.

11. A computing device comprising:
a processor;
a memory coupled to the processor; and
instructions provided to the memory, wherein the instructions are executable by the processor to perform the method of claim 1.

12. A method comprising:
determining a source facies for a hydrocarbon sample;
measuring a concentration or a related value of age biomarkers in the hydrocarbon sample to yield an age biomarker fingerprint;
inputting the source facies and the age biomarker fingerprint into a chemical fossil assemblage model;
comparing the age biomarker fingerprint to one or more facies-dependent chemical fossil assemblages using the chemical fossil assemblage model; and
estimating an age of the hydrocarbon sample based on the comparison.

13. The method of claim 12, wherein the source facies is selected from the group consisting of: a marine carbonate/marly facie, a marine clastic/deltaic facie, a terrestrial/coaly facie, and a lacustrine facie.

14. The method of claim 12, wherein the chemical fossil assemblage model is a predictive model trained using a database of known sample ages and age biomarker compositions.

15. The method of claim 12, wherein the measuring of the concentration or the related value of the age biomarkers:
separating the hydrocarbon sample into separated fractions including an aromatics fraction and a saturates fraction;
analyzing the saturates fraction using one or more of: gas chromatography-isotope ratio mass spectrometry, gas chromatography/mass spectrometry, and gas chromatography/mass spectrometry tandem mass spectrometry; and
analyzing the aromatics fraction using gas chromatography-mass spectrometry.

16. The method of claim 12, further comprising:
performing one or more analyses selected from: time of flight mass spectroscopy, whole oil gas chromatography, ¹³C isotopic composition, full-scan gas chromatography/mass spectroscopy, infrared-gas chromatography/mass spectroscopy, and C₄ to C₁₉ gas chromatography.

17. The method of claim 12, further comprising:
estimating risk for drilling a wellbore and producing hydrocarbon from the wellbore in the location from which the hydrocarbon sample was obtained based on the age of the hydrocarbon sample and a thermal history of the location from which the hydrocarbon sample was obtained.

18. The method of claim 12, further comprising:
drilling a wellbore into a location from which the hydrocarbon sample was obtained.

19. The method of claim 12, further comprising:
producing hydrocarbon from a location from which the hydrocarbon sample was obtained.

20. A computing device comprising:
a processor;
a memory coupled to the processor; and
instructions provided to the memory, wherein the instructions are executable by the processor to perform the method of claim 12.
